(19) United States
(12) Patent Application Publication
(10) Pub. No.: US 2023/0193347 A1
(43) Pub. Date: Jun. 22, 2023

(54) Biological Indicator with Test Microorganisms Enveloped by Wax Composition

(71) Applicant: 3M INNOVATIVE PROPERTIES COMPANY, St. Paul, MN (US)

(72) Inventors: Ahimou Francois, Woodbury, MN (US); Timothy A. Kohman, Cottage Grove, MN (US); Sarah J. Davis, Stillwater, MN (US); Michael E. Benz, Ramsey, MN (US)

(21) Appl. No.: 17/995,869

(22) PCT Filed: Apr. 13, 2021
(86) PCT No.: PCT/IB2021/053031

§ 371 Date: Oct. 10, 2022

Related U.S. Application Data
(60) Provisional application No. 63/013,782, filed on Apr. 22, 2020.

(51) Int. Cl. C12Q 1/22; A61L 2/28

(52) U.S. Cl. CPC C12Q 1/22; A61L 2/28

(57) ABSTRACT

A self-contained sterilization process biological indicator is provided. The indicator includes a housing. Test microorganisms disposed in the housing are enveloped by a wax composition. The wax composition comprises a long-chain (greater than C22) linear or branched alkyl or alkenyl alcohol, wherein the alkyl or alkenyl alcohol is not linked through an ester bond or an ether bond to a short chain alkyl or aryl (C1-C6) group, wherein the alkyl or alkenyl alcohol is not linked through an ester bond or an ether bond to a long chain (C8-C36) alkyl or alkenyl group, wherein the alkyl or alkenyl alcohol is not linked through an amide bond to a linear or branched long chain (C8-C36) alkyl or alkenyl amine or acid. The wax composition has a melting point between 78° C. and 120° C. Also contained in the housing are a nutrient composition and an openable container containing a liquid medium.

### BACKGROUND

The sterilization of equipment, instruments, and other devices is critical in the health care industry. For example, hospitals and other medical institutions frequently sterilize medical instruments and equipment used in treating patients. The particular type of sterilization cycle used to sterilize such equipment can vary based on the particular equipment or devices being sterilized and based on the particular preference of the entity performing the sterilization cycle. However, all such sterilization cycles or processes are typically designed to kill living organisms which might otherwise contaminate the equipment or devices being sterilized.

Various sterilization methods use different cycles or techniques for sterilization. For instance, sterilization may include the administration of steam, dry heat, chemicals (e.g., ethylene oxide, hydrogen peroxide), or radiation, to the equipment or devices being sterilized. Steam sterilization is typically recommended for metal, Teflon, and other high melting point surgical instruments capable to hold a temperature range of 121-135° C. The exposure time to in a sterilization cycle is temperature dependent. For example, for equipment or instruments being sterilized are preferably exposed to the steam sterilization for approximately three minutes at 132° C. However, the exposure period could be up to 30-35 minutes at 121° C.

Biological indicators are commonly used to evaluate and validate the effectiveness of a sterilization process in a variety of settings. In general, viable but relatively highly-resistant spores of thermophilic organisms are subjected to the sterilization conditions along with any devices or instruments to be sterilized. In general, the test microorganisms are more resistant to the sterilization process than most other organisms that would be present by natural contamination. Applications have used spores of microorganisms capable of producing an enzyme that catalyzes the reaction of a non-fluorescent substrate to a fluorescent product that can be detected to indicate the presence of surviving spores.

Typically, after completion of the sterilization process, the test microorganisms (e.g., spores) are incubated in nutrient medium to determine whether any of the test organisms survived the sterilization procedure. In the conventional biological indicators, growth of a detectable number of organisms can take 24 hours or more when using a pH indicator to detect the growth.

Rapid readout technology, using detection of test microorganism-associated enzyme activity, can reduce the time necessary to detect viable test microorganisms. In some implementations, an analysis of the fluorescence intensity due to a fluorescent product of an enzyme reaction serves to determine whether the sterilization process was successful.

### SUMMARY

A self-contained sterilization process biological indicator is now provided, therein with everything needed to rapidly assess the effectiveness of a variety of steam sterilization processes by enabling the detection of germination and/or outgrowth of viable test microorganisms, if present, after exposing the self-contained biological indicator to a steam sterilization process. Advantageously, this discovery provides its user with a biological indicator that has test microorganisms enveloped by a wax composition that increases the resistance of the test microorganisms to certain steam sterilization processes, thereby providing biological indicators that are useful to assess the efficacy of a wide range of steam sterilization process conditions. Surprisingly, the wax composition-enveloped test microorganisms of the present disclosure are suitable both for rapid detection (e.g., by detecting an enzyme activity associated with the test microorganisms) and for traditional, microorganism growth-based detection.

In one aspect, the present disclosure provides a self-contained sterilization process biological indicator. The indicator can comprise a housing having at least one liquid-impermeable wall that forms an opening into a compartment; a plurality of test microorganisms disposed in the housing, wherein the test microorganisms are at least partially enveloped by a wax composition; a liquid medium disposed in an openable container, the contents of the container being in selective fluid communication with the compartment; and a nutrient composition that facilitates germination and/or outgrowth of the test microorganisms, wherein the nutrient composition is disposed in the container or the housing. The wax composition comprises a long-chain (greater than C22) linear or branched alkyl or alkenyl alcohol, wherein the alkyl or alkenyl alcohol is not linked through an ester bond or an ether bond to a short chain alkyl or aryl (C1-C6) group, wherein the alkyl or alkenyl alcohol is not linked through an ester bond or an ether bond to a long chain (C8-C36) alkyl or alkenyl group, wherein the alkyl or alkenyl alcohol is not linked through an amide bond to a linear or branched long chain (C8-C36) alkyl or alkenyl amine or acid. The wax composition has a melting point between 78° C. and 120° C. The housing comprises an opening that permits passage of a sterilant, from outside the housing into the housing.

In certain embodiments of the self-contained sterilization process biological indicator, the test microorganisms can be affixed to a carrier, wherein the test microorganisms are enveloped by the wax composition and/or the carrier. In certain embodiments of the self-contained sterilization process biological indicator, the test microorganisms can be affixed to a portion of the wall of the housing, wherein the test microorganisms are enveloped by the wax composition and/or the portion of the wall. In any of the above embodiments, the self-contained biological indicator for sterilization process further can comprise a wax composition-wicking member. In certain embodiments, the wax composition-wicking member can contact the wax composition.

In another aspect, the present disclosure provides a method of determining effectiveness of a sterilization process. The method can comprise positioning a sterilization process biological indicator in a sterilization chamber, wherein the biological indicator comprises a plurality of test microorganisms that are at least partially enveloped by a wax composition; while the indicator is positioned in the sterilization chamber, exposing the biological indicator to a moist heat at a temperature of at least 121° C.; after exposing the biological indicator to the moist heat, contacting the test microorganisms with a detection medium; and after contacting the test microorganisms with the detection medium, incubating the indicator at a predetermined temperature for a period of time sufficient to detect a presence of one of the test microorganisms, if viable. The wax composition comprises a long-chain (greater than C22) linear or branched alkyl or alkenyl alcohol, wherein the alkyl or alkenyl alcohol is not linked through an ester bond or an ether bond to a short chain alkyl or aryl (C1-C6) group, wherein the alkyl or alkenyl alcohol is not linked through an ester bond or an ether bond to a long chain (C8-C36) alkyl or alkenyl group, wherein the alkyl or alkenyl alcohol is not linked through an amide bond to a linear or branched long chain (C8-C36) alkyl or alkenyl amine or acid. The wax composition has a melting point between 78° C. and 120° C.

In any of the above embodiments of the method, positioning a sterilization process biological indicator in a sterilization chamber can comprise positioning a sterilization process biological indicator comprising a housing with the test microorganisms therein, wherein contacting the test microorganisms with a detection medium can comprise a contacting the test microorganisms with the detection medium inside the housing.

In yet another aspect, the present disclosure provides a kit. The kit can comprise a plurality of test microorganisms, wherein the test microorganisms are at least partially enveloped by a wax composition, and instructions for using the test microorganisms to assess the efficacy of a steam sterilization process. The wax composition comprises a long-chain (greater than C22) linear or branched alkyl or alkenyl alcohol, wherein the alkyl or alkenyl alcohol is not linked through an ester bond or an ether bond to a short chain alkyl or aryl (C1-C6) group, wherein the alkyl or alkenyl alcohol is not linked through an ester bond or an ether bond to a long chain (C8-C36) alkyl or alkenyl group, wherein the alkyl or alkenyl alcohol is not linked through an amide bond to a linear or branched long chain (C8-C36) alkyl or alkenyl amine or acid. The wax composition has a melting point between 78° C. and 120° C.

In any embodiment, the kit further can comprise a detection reagent selected from the group consisting of a nutrient, a pH indicator, a redox indicator, a fluorogenic enzyme substrate, a chromogenic enzyme substrate, and a combination of any two or more of the foregoing detection reagents. In any of the above embodiments, the kit further can comprise a housing dimensioned to contain the test microorganisms or the detection reagent. In certain embodiments of the kit, the test microorganisms and the detection reagent can be disposed in the housing.

Additional details of these and other embodiments are set forth in the accompanying drawings and the description below. Other features, objects and advantages will become apparent from the description and drawings, and from the accompanying claims.

Herein, the terms “biological sterilization process indicators”, “sterilization process biological indicator”, “sterilization process indicator”, “biological indicator”, “BI”, “indicator”, “self-contained biological indicator”, and “SCBI” are used interchangeably.

Also herein, in the written description and the claims, the phrases “substantially dry”, “substantially water-free” or the like refer to a composition or a coating which has a water content no greater than about the water content of the dehydrated coating once it has been permitted to equilibrate with the ambient environment.

The numbers, E5, E6, and E7 are used interchangeably herein with 10⁵, 10⁶, and 10⁷, respectively.

The term “comprising” and variations thereof (e.g., comprises, includes, etc.) do not have a limiting meaning where these terms appear in the description and claims.

As used herein, “a”, “an”, “the”, “at least one”, and “one or more” are used interchangeably, unless the context clearly dictates otherwise.

Also herein, the recitations of numerical ranges by endpoints include all numbers subsumed within that range (e.g., 500 to 7000 nm includes 500, 530, 551, 575, 583, 592, 600, 620, 650, 700, etc.).

The words “preferred” and “preferably” refer to embodiments of the invention that may afford certain benefits, under certain circumstances. However, other embodiments may also be preferred, under the same or other circumstances. Furthermore, the recitation of one or more preferred embodiments does not imply that other embodiments are not useful and is not intended to exclude other embodiments from the scope of the invention.

The term “powerset” as used herein for a given set S having n elements refers to the mathematical definition of a powerset and all possible subsets of S, without including the empty set, but including S itself, having from 1 to n elements in every combination and is denoted as P(S). Applicants note that the mathematical definition of a powerset includes the empty set (a set having no elements). However, the definition adopted here by Applicants excludes the empty set and includes all subsets having at least one element, including the full set of n elements (S). In general, the powerset includes all subsets having “i” elements for i=1 to n−1, and the subset having all n elements (n). For instance, the powerset of a subset S having the elements a, b, and c (n=3) includes the following 7 subsets: all possible subsets having one element: {(a), (b), (c)}; all possible subsets having any possible combination two elements: {(a, b), (a, c), (b, c)}, and the subset having all 3 elements: (a, b, c).

The term “actuatable container”, as used herein, refers to a container that can be actuated, when desired, to release contents therein. The container can be actuated, for example, the container can by dislodging or removing a plug, by actuating a valve to change it from a “closed” state to an “open” state, or by otherwise breaching at least a portion of the container.

The term “frangible container” refers to any container that can be acted upon to release its contents, for example by breaking it, puncturing it, shattering it, cutting it, etc.

All scientific and technical terms used herein have meanings commonly used in the art unless otherwise specified. The definitions provided herein are to facilitate understanding of certain terms used frequently in this application and are not meant to exclude a reasonable interpretation of those terms in the context of the present disclosure.

Unless otherwise indicated, all numbers in the description and the claims expressing feature sizes, amounts, and physical properties used in the specification and claims are to be understood as being modified in all instances by the term “about.” Accordingly, unless indicated to the contrary, the numerical parameters set forth in the foregoing specification and attached claims are approximations that can vary depending upon the desired properties sought to be obtained by those skilled in the art utilizing the teachings disclosed herein. At the very least, and not as an attempt to limit the application of the doctrine of equivalents to the scope of the claims, each numerical parameter should at least be construed in light of the number of reported significant digits and by applying ordinary rounding techniques. Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviations found in their respective testing measurements.

The term “adjacent” refers to the relative position of two elements, such as, for example, two layers, that are close to each other and may or may not be necessarily in contact with each other or that may have one or more layers separating the two elements as understood by the context in which “adjacent” appears.

The above summary of the present invention is not intended to describe each disclosed embodiment or every implementation of the present invention. The description that follows more particularly exemplifies illustrative embodiments.

### BRIEF DESCRIPTION OF DRAWINGS

FIG. 1 is a partially-exploded cross-sectional view of a self-contained biological indicator of the present disclosure.

FIG. 2 is a cross-sectional view, along line 2-2, of the self-contained biological indicator of the FIG. 1.

FIG. 3 is an exploded perspective view of the self-contained biological indicator of the FIGS. 1-2.

FIG. 4A is a schematic plan view of one embodiment of a test microorganism carrier to which test microorganisms enveloped by a wax composition are affixed according to the present disclosure.

FIG. 4B is a cross-sectional view, along line 4B-4B, of the test microorganism carrier of FIG. 4A

FIG. 5 is a cross-sectional view of an alternative embodiment of a housing of a self-contained biological indicator according to the present disclosure, wherein the microorganisms enveloped by a wax composition are affixed to at least one wall of the housing.

### DETAILED DESCRIPTION

Before any embodiments of the present disclosure are explained in detail, it is to be understood that the invention is not limited in its application to the details of construction and the arrangement of components set forth in the following description or illustrated in the following drawings. The invention is capable of other embodiments and of being practiced or of being carried out in various ways. Also, it is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items. It is to be understood that other embodiments may be utilized, and structural or logical changes may be made without departing from the scope of the present disclosure. Furthermore, terms such as “front,” “rear,” “top,” “bottom,” and the like are only used to describe elements as they relate to one another, but are in no way meant to recite specific orientations of the apparatus, to indicate or imply necessary or required orientations of the apparatus, or to specify how the invention described herein will be used, mounted, displayed, or positioned in use.

The present disclosure generally relates to sterilization process indicators, kits containing test microorganisms for use as sterilization process indicators, and methods of use thereof. A self-contained biological indicator of the present disclosure comprises all of the components necessary to assess the survival of a test microorganism contained therein and can be used to determine the lethality of a sterilizing process that uses moist heat as the sterilant.

As indicated above, self-contained biological sterilization process indicators are now provided which can be used to assess the efficacy of a variety of steam sterilization processes including, for example, steam sterilization processes that employ temperatures at 121° C., 132° C., 134° C., or 135° C. Advantageously, this means the operator no longer must use a different biological indicator for each of a plurality of different steam sterilization temperatures.

Housing

For non-limiting examples of housings suitable for use in self-contained biological indicators, please see U.S. Pat. Nos. 3,661,717; 5,223,401 and 6,623,955; and U.S. Patent Application Publication Nos. 2013/0302849 and 2014/0349335; each of which is incorporated herein by reference in its entirety. In general, the housing refers to a container, usually an outer container, having walls impermeable to a sterilant, where other components of the biological indicator are located. The housing may be inside a process challenge device or may be a process challenge device itself. In some embodiments, the housing may have dimensions useful to produce a flat or generally planar biological indicator. This disclosure encompasses housings of any shape and dimensions.

The housing contains at least one opening that allows flow of a sterilant to the interior of the housing (sterilant pathway). In some embodiments, the housing may comprise a body with an opening and a cap to close that opening. In some embodiments, the cap may be capable of completely sealing the housing and eliminating any fluid communication between the interior of the housing and ambiance (e.g., closing the sterilant pathway). In general, the cap has an open position in which there is an opening (e.g., a gap) between the cap and the body of the container that allows flow of liquid or gas (e.g., a sterilant) into and out of the interior of the housing. The cap also has a closed position where the opening is sealed and any fluid flow through the gap is eliminated. In other embodiments, the cap may comprise vents that allow passage of a sterilant to the interior of the housing and create an additional sterilant pathway, even if the cap is present and in the closed position. In other preferred embodiments, however, when the cap comprises vents, placing the cap in the closed position simultaneously closes: (a) the gap between the cap and the body of the container and (b) the vents present on the cap, essentially closing the sterilant pathway.

In other embodiments, the cap may lack vents and the only sterilant pathway may be through the space between the cap and the body of the housing (or through another opening or vent, if present on the body) when the cap is the open position. In some embodiments, if vents exist on the housing, they are located on the cap. In embodiments where no other opening exists besides the opening between the cap and the body of the housing, then placing the cap in the closed position completely seals off the interior of the housing, which stops the fluid communication between the interior of the housing and ambience. In those embodiments, the sterilant pathway may be sealed when the cap is in the closed position.

Test Microorganisms

Generally, test microorganisms are chosen to be used in a biological indicator that are particularly resistant to a given sterilization process. In certain embodiments, the biological indicators of the present disclosure include a viable culture of a known species of microorganism, usually in the form of microbial spores. Spores (e.g., bacterial spores), rather than the vegetative form of the microorganisms, are used at least partly because vegetative microorganisms are known to be relatively easily killed by sterilizing processes. Additionally, spores also have superior storage characteristics and could remain in their dormant state for years. As a result, sterilization of an inoculum of a standardized spore strain provides a higher degree of confidence that inactivation of all microorganisms in a sterilizing chamber has occurred.

A self-contained biological indicator of the present disclosure includes a plurality of test microorganisms disposed therein (e.g., disposed in the interior of the housing). The test microorganisms may be of one or more species. Typically, the biological indicator contains a predetermined quantity of at least 10³, at least 10⁴, at least 10⁵, at least 10⁶, at least 10′, or at least 10⁸ test microorganisms

By way of example only, the present disclosure describes the microorganisms used in the biological indicator as being “spores;” however, it should be understood that the type of microorganism (e.g., spore) used in a particular embodiment of the biological indicator is selected for being resistant to the particular sterilization process contemplated (more resistant than the microorganisms normally present on the items to be sterilized so that inactivation of the test microorganisms indicates a successful sterilization.). Accordingly, different embodiments of the present disclosure using different sterilants may use different microorganisms, depending on the sterilization process for which the particular embodiment is intended.

In some embodiments, the test microorganisms can include, but are not limited to, at least one of Geobacillus stearothermophilus, Bacillus stearothermophilus, Bacillus subtilis, Bacillus atrophaeus, Bacillus megaterium, Bacillus coagulans, Clostridium sporogenes, Bacillus pumilus, or combinations thereof

Enzymes and Enzyme Substrates

A self-contained biological indicator of the present disclosure comprises a detection reagent (e.g., a nutrient that facilitates germination and/or outgrowth of the test microorganism). In some embodiments, the biological indicator may comprise a detection reagent (e.g., an enzyme substrate) capable of detecting an enzyme present in and/or on the test microorganisms, or the test microorganisms are capable of producing such an enzyme, or both. The enzymes useful in biological indicators of the present disclosure include extracellular and intracellular enzymes whose activity correlates with the viability of at least one of the microorganisms commonly used to monitor sterilization efficacy (“test” microorganism or “test spores”). In this context, “correlates” means that the enzyme activity, over background, can be used to demonstrate survival of at least one of the test microorganisms. The enzyme should be one which, following a sterilization cycle which is sublethal to the test microorganism, remains sufficiently active to react with an enzyme substrate for the enzyme, within twenty-four hours, and in preferred embodiment within one hours or less, yet be inactivated or appreciably reduced in activity following a sterilization cycle which would be lethal to the test microorganism.

Examples of suitable enzymes include α-glucosidase, α-galactosidase, lipase, esterase, acid phosphatase, alkaline phosphatase, proteases, aminopeptidase, chymotrypsin, β-glucosidase, β-galactosidase, α-glucoronidase, β-glucoronidase, phosphohydrolase, α-mannosidase, β-mannosidase, a-L-fucosidase, leucine aminopeptidase, a-L-arabinofuranoside, cysteine aminopeptidase, valine aminopeptidase, β-xylosidase, α-L-iduronidase, glucanase, cellobiosidase, cellulase, α-arabinosidase, glycanase, sulfatase, butyrate, glycosidase, arabinosidase, and a combination of any two or more of the foregoing enzymes. In certain embodiments of the articles, kits, systems and methods of the present disclosure, the source of biological activity used therein comprises an isolated or otherwise purified form of any of the foregoing suitable enzymes.

In the context of this application, an enzyme substrate comprises a substance or mixture of substances that, when acted upon by an enzyme, are converted into an enzyme-modified product. Although the preferred substrate produces a fluorescently detectable compound, in other embodiments, the product of the enzymatic action may be a luminescent or colored material. In other embodiments, however, the enzyme substrate can consist of a compound which when reacted with the enzyme, will yield a product that will react with an additional compound or composition to yield a luminescent, fluorescent, or colored material. Preferably, if the substrate is to be included in the indicator device during sterilization, the substrate should not spontaneously break down or convert to a detectable product during sterilization or incubation. For example, in devices used to monitor steam and dry heat sterilization, the substrate must be stable at temperatures between about 20° C. and 180° C. Preferably also, where the enzyme substrate is to be included with conventional growth media, it must be stable in the growth media, e.g., not auto fluoresce in the growth media.

In general, there are two basic types of enzyme substrate that can be used in the biological indicators of this disclosure. The first type of substrate can be either fluorogenic (or chromogenic), and can be given a chemical formula such as, AB. When acted upon by the enzyme, AB breaks down into the products A and B. B, for example, could be either fluorescent or colored. A specific example of a fluorogenic substrate of this type are salts of 4-methylumbelliferyl. Other fluorogenic substrates of this type include the derivatives of 4-methylumbelliferyl, 7-amido-4-methylcoumarin (7-AMC), indoxyl and fluorescein. An example of a chromogenic substrate of this type is 5-bromo-4-chloro-3-indolyl phosphate. In the presence of phosphatase, the substrate will be broken down into indigo blue and phosphate. Other chromogenic substrates of this type include derivatives of 5-bromo-4-chloro-3-indolyl, nitrophenol and phenolphthalein, listed below.

The second type of substrate can be given the chemical formula CD, for example, which will be converted by a specific enzyme into C and D. In this case, however, neither C nor D will be fluorescent or colored, but either C or D is capable of being further reacted with compound Z to give a fluorescent or colored compound, thus indicating enzyme activity. A specific fluorogenic example of this type is the amino acid lysine. In the presence of the enzyme lysine decarboxylase, lysine loses a molecule of CO₂. The remaining part of the lysine is then called cadaverine, which is strongly basic. A basic indicator such as 4-methylumbelliferone can be incorporated and will fluoresce in the presence of a strong base. A chromogenic substrate of this type would be 2-naphthyl phosphate. The enzyme phosphatase reacts with the substrate to yield beta-naphthol. The liberated beta-naphthol reacts with a chromogenic reagent containing 1-diazo-4-benzoylamino-2, 5-diethoxybenzene, commercially available as “Fast Blue BB Salt” from Sigma Chemical, to produce a violet color.

As mentioned above, a preferred enzyme substrate in some embodiments is a fluorogenic substrate, defined herein as a compound capable of being enzymatically modified, e.g., by hydrolysis or other enzymatic action, to give a derivative fluorophore that has a measurably modified or increased fluorescence.

A person having ordinary skill in the art would understand that suitable fluorogenic compounds are in themselves either non-fluorescent or meta-fluorescent (i.e., fluorescent in a distinctly different way e.g., either by color or intensity, compared to the corresponding enzyme-modified products). In that regard, appropriate wavelengths of excitation and detection, in a manner known to users of fluorometric techniques, are used to separate the fluorescence signal developed by the enzyme modification from any other fluorescence that may be present.

Non-limiting examples of suitable enzymatic substrates can include, for example, derivatives of coumarin including 7-hydroxycoumarin (also known as umbelliferone or 7-hydroxy-2H-chromen-2-one) derivatives and 4-methylumbelliferone (7-hydroxy-4-methylcoumarin) derivatives including: 4-methylumbelliferyl alpha-D-glucopyranoside, 4-methylumbelliferyl alpha-D-galactopyranoside, 4-methylumbelliferyl heptanoate, 4-methylumbelliferyl palmitate, 4-methylumbelliferyl oleate, 4-methylumbelliferyl acetate, 4-methylumbelliferylnonanoate, 4-methylumbelliferyl caprylate, 4-methylumbelliferyl butyrate, 4-methylumbelliferyl-beta-D-cellobioside, 4-methylumbelliferyl acetate, 4-methylumbelliferyl phosphate, 4-methylumbelliferyl sulfate, 4-methylumbelliferyl-beta-trimethylammonium cinnamate chloride, 4-methylumbelliferyl-beta-D-N, N′,N″-triacetylchitotriose, 4-methylumbelliferyl-beta-D-xyloside, 4-methylumbelliferyl-N-acetyl-beta-D-glucosaminide, 4-methylumbelliferyl-N-acetyl-alpha-D-glucosaminide, 4-methylumbelliferyl propionate, 4-methylumbelliferyl stearate, 4-methylumbelliferyl-alpha-L-arabinofuranoside, 4-methylumbelliferyl alpha-L-arabinoside;methyl umbelliferyl-beta-D-N,N′-diacetyl chitobioside, 4-methylumbelliferyl elaidate, 4-Methylumbelliferyl-alpha-D-mannopyranoside, 4-methylumbelliferyl-beta-D-mannopyranoside, 4-methylumbelliferyl-beta-D-fucoside, 4-methylumbelliferyl-alpha-L-fucoside, 4-methylumbelliferyl-beta-L-fucoside, 4-methylumbelliferyl-alpha-D-galactoside, 4-methylumbelliferyl-beta-D-galactoside, 4-trifluoromethylumbelliferyl beta-D-galactoside, 4-methylumbelliferyl-alpha-D-glucoside, 4-methylumbelliferyl-beta-D-glucoside, 4-methylumbelliferyl-7,6-sulfo-2-acetamido-2-deoxy-beta-D-glucoside, 4-methylumbelliferyl-beta-D-glucuronide, 6,8-difluor-4-methylumbelliferyl-beta-D-glucuronide, 6,8-difluoro-4-methylumbelliferyl-beta-D-galactoside, 6,8-difluoro-4-methylumbelliferyl phosphate, 6,8-difluoro-4-methylumbelliferyl beta-D-xylobioside, for example. The second substrate can also be derivatives of 7-amido-4-methylcoumarin, including: Ala-Ala-Phe-7-amido-4-methylcoumarin, Boc-Gln-Ala-Arg-7-amido-4-methylcoumarin hydrochloride, Boc-Leu-Ser-Thr-Arg-7-amido-4-methylcoumarin, Boc-Val-Pro-Arg-7-amido-4-methylcoumarin hydrochloride, D-Ala-Leu-Lys-7-amido-4-methylcoumarin, L-Alanine 7-amido-4-methylcoumarin trifluoroacetate salt, L-Methionine 7-amido-4-methylcoumarin trifluoroacetate salt, L-Tyrosine 7-amido-4-methylcoumarin, Lys-Ala-7-amido-4-methylcoumarin dihydrochloride, N-p-Tosyl-Gly-Pro-Arg 7-amido-4-methylcoumarin hydrochloride, N-Succinyl-Ala-Ala-Phe-7-amido-4-methylcoumarin, N-Succinyl-Ala-Ala-Pro-Phe-7-amido-4-methylcoumarin, N-Succinyl-Ala-Phe-Lys 7-amido methylcoumarin acetate salt, N-Succinyl-Leu-Leu-Val-Tyr-7-Amido-4-methylcoumarin, D-Val-Leu-Lys 7-amido-4-methylcoumarin, Fmoc-L-glutamic acid 1-(7-amido-4-methylcoumarin), Gly-Pro amido-4-methylcoumarin hydrobromide, L-Leucine-7-amido-4-methylcoumarin hydrochloride, L-Proline-7-amido-4-methylcoumarin hydrobromide; other 7-hydroxycoumarin derivatives including 3-cyano-7-hydroxycoumarin β-cyanoumbelliferone), and 7-hydroxycoumarin-3-carboxylic acid esters such as ethyl-7-hydroxycoumarin-3-carboxylate, methyl-7-hydroxycoumarin-3-carboxylate, 3-cyano-4-methylumbelliferone, 3-(4-imidazolyl)umbelliferone; derivatives of fluorescein including: 2′,7′-Bis-(2-carboxyethyl)-5-(and-6-)carboxyfluorescein, 2′,7′-bis-(2-carboxypropyl)-5-(and-6+carboxyfluorescein, 5- (and 6)-carboxynaphthofluorescein, Anthofluorescein, 2′,7′-Dichlorofluorescein diacetate, 5(6)-Carboxyfluorescein, 5(6)-Carboxyfluorescein diacetate, 5-(Bromomethyl)fluorescein, 5-(Iodoacetamido)fluorescein, 5-([4,6-Dichlorotriazin-2-yl]amino)fluorescein hydrochloride, 6-Carboxyfluorescein, Eosin Y, Fluorescein diacetate 5-maleimide, Fluorescein-O′-acetic acid, O′-(Carboxymethyl)fluoresceinamide, anthofluorescein, rhodols, halogenated fluorescein; derivatives of rhodamine including: Tetramethylrhodamine, Carboxy tetramethyl-rhodamine, Carboxy-X-rhodamine, Sulforhodamine 101 and Rhodamine B; afluorescamine derivatives; derivatives of benzoxanthene dyes including: seminaphthofluorones, carboxy-seminaphthofluorones seminaphthofluoresceins, seminaphthorhodafluors; derivatives of cyanine including sulfonated pentamethine and septamethine cyanine.

In some embodiments, the enzyme whose activity is to be detected may be chosen from alpha-D-glucosidase, chymotrypsin, or fatty acid esterase. In the case of Bacillus stearothermophilus, the fluorogenic enzyme substrate is preferably 4-methylumbelliferyl-alpha-D-glucoside, 7-glutarylphenylalanine-7-amido-4-methyl coumarin, or 4-methylumbelliferyl heptanoate. In certain preferred embodiments, 4-methylumbelliferyl alpha-D-glucopyranoside is the enzyme substrate used to produce the metabolic activity and the enzyme is a glucosidase, such as beta-D-glucosidase.

The concentration of enzyme substrate present in the biological indicator (e.g., when dissolved and/or suspended in aqueous liquid medium in the biological indicator) depends upon the identity of the particular enzyme substrate and enzyme, the amount of enzyme-product that must be generated to be detectable, either visually or by instrument, and the amount of time that one is willing to wait in order to determine whether active enzyme is present in the reaction mixture. Preferably, the amount of enzyme substrate is sufficient to react with any residual active enzyme present, after the sterilization cycle, within about an eight-hour period of time, such that at least 10⁻⁸ molar enzyme-modified product is produced. In cases where the enzyme substrate is a 4-methylumbelliferyl derivative, the inventors have been found that its concentration in the aqueous liquid medium disclosed herein is preferably between about 10⁻⁵ and 10⁻³ molar. In some embodiments, the 4-methylumbelliferyl-α-D-glucoside can be used, for example, at a concentration of about 0.05 to about 0.5 g/L (e.g., about 0.05 g/L, about 0.06 g/L, about 0.07 g/L, about 0.08 g/L, about 0.09 g/L, about 0.1 g/L, about 0.15 g/L, about 0.2 g/L, about 0.25 g/L, about 0.3 g/L, about 0.35 g/L, about 0.4 g/L, about 0.45 g/L, about 0.5 g/L) in the aqueous mixture.

pH Indicator Dye

In any embodiment, a self-contained biological indicator of the present disclosure can comprise a pH indicator dye disposed in the housing (e.g., in the compartment). In certain embodiments, the pH indicator day can be bound (e.g., with high affinity) to a pH indicator dye substrate material as described in U.S. Provisional Patent Application No. 62/990,483; filed on Mar. 17, 2020 and entitled “IMMOBILIZED PH INDICATOR FOR BIOLOGICAL INDICATOR GROWTH INDICATION”, which is incorporated herein by reference in its entirety. In any embodiment, the indicator dye may be a pH indicator suitable to detect biological activity (e.g., fermentation of a carbohydrate nutrient). The indicator dye can be selected according to criteria known in the art such as, for example, pH range, compatibility with the biological activity, and solubility. In some embodiments, a salt form of the pH indicator may be used, for example, to increase the solubility of the pH indicator in an aqueous mixture. Nonlimiting examples of suitable pH indicator dyes include, for example, thymol blue, tropeolin OO, methyl yellow, methyl orange, bromophenol blue, bromocresol green, methyl red, bromothymol blue, phenol red, chlorophenol red, neutral red, naphtholphthalein, phenolphthalein, thymolphthalein, alizarin yellow, tropeolin O, nitramine, trinitrobenzoic acid, thymol blue, bromophenol blue, tetrabromphenol blue, bromocresol green, bromocresol purple, methyl red, bromothymol blue, Congo red, and cresol red. In certain embodiments, the pH indicator dye is anionic in a solution having a pH around neutral.

In some embodiments, the pH indicator dye produces a change in color when the pH decreases, indicating growth of the test microorganisms. In some embodiments, the pH indicator dye is bromocresol purple. The pH indicator can be used to detect a biological activity, such as the fermentation of a carbohydrate to acid end products (suggesting survival of the test microorganisms). These activities can indicate the presence or absence of a viable spore following the exposure of a biological indicator to a sterilization process, for example. The bromocresol purple can be used at a concentration of about 0.03 g/L in the aqueous mixture, for example.

The combination of bromocresol purple and 4-methylumbelliferyl-α-D-glucoside represents a preferred combination of enzymatic substrate and pH indicator dye in an article or method according to the present disclosure, but other combinations are contemplated within the scope of the present disclosure.

Self-Contained Biological Indicator

The plurality of test microorganisms enveloped by a wax composition described herein can be employed as a modification to a wide variety of biological indicators known in the art to produce a biological indicator or a self-contained biological indicator according to the present disclosure. The resulting biological indicator or self-contained biological indicator is particularly useful for assessing the effectiveness of a steam sterilization process. In addition, the plurality of test microorganisms enveloped by a wax composition described herein can be employed as a modification to a wide variety of methods of assessing the effectiveness of a sterilization process.

For example, the self-contained biological indicator of U.S. Pat. No. 3,661,717; which is incorporated herein by reference in its entirety; could be modified to provide the test microorganisms enveloped by a wax composition as described herein. In certain embodiments, the test microorganisms enveloped by the wax composition could be provided on a carrier substrate or on an inner surface of the housing of the biological indicator.

In addition, the self-contained biological indicators of U.S. Pat. Nos. 5,223,401 and 6,623,955; which are both incorporated herein by reference in their entirety; could be modified to provide the test microorganisms enveloped by a wax composition as described herein. In certain embodiments, the test microorganisms enveloped by the wax composition could be provided on a carrier substrate or on an inner surface of the housing of the biological indicator.

Furthermore, the self-contained biological indicator of U.S. Patent Application Publication No. US 2013/0302849; which is incorporated herein by reference in its entirety; could be modified to provide the test microorganisms enveloped by a wax composition as described herein. In certain embodiments, the test microorganisms enveloped by the wax composition could be provided on a carrier substrate or on an inner surface of the housing of the biological indicator.

A person having ordinary skill in the art will recognize how other existing biological indicators could be modified with the test microorganisms enveloped by a wax composition of the present disclosure to arrive at the articles and methods of the present disclosure.

In this disclosure, the process of bringing the spores and medium together is referred to as “activation” of the biological indicator. That is, the term “activation” and variations thereof, when used with respect to a biological indicator refer generally to bringing one or more test microorganisms (e.g., spores) in fluid communication with the aqueous liquid medium (e.g., a liquid medium comprising a nutrient and/or an enzyme substrate). For example, when an openable container within the biological indicator that contains the aqueous liquid medium is at least partially opened (e.g., fractured, punctured, pierced, crushed, cracked, breaking, or the like), such that the medium has been put in fluid communication with the test microorganisms, the biological indicator can be described as having been “activated.”

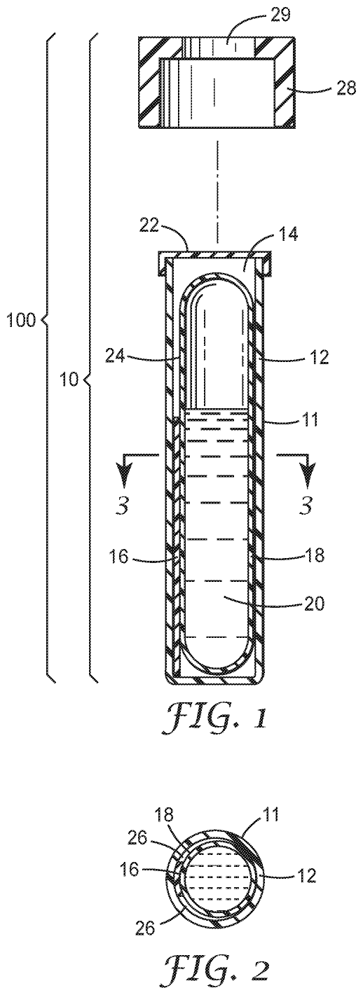

Turning now to the drawings, FIGS. 1-3 show various views of one embodiment of a self-contained biological indicator 100 according to the present disclosure.

The self-contained biological indicator 100 is shown as having a housing 10 that comprises a compartment 11 and a cap 28. The compartment 11 has at least one wall 12 that forms an opening 14. The at least one wall can be made of a moisture-impermeable, nonabsorptive material such as glass or plastic, for example. In certain preferred embodiments, the compartment is formed from an optically transparent or translucent material.

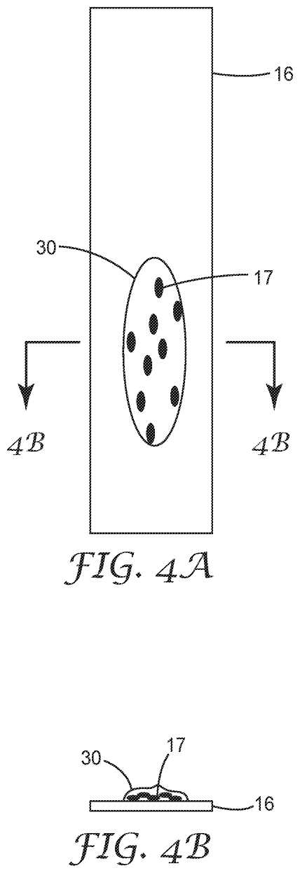

The biological indicator 100 contains a plurality of test microorganisms (e.g., bacterial spores) 17 disposed in the housing 10. The test microorganisms 17 optionally can be disposed on a carrier 16 (e.g., a sheet-like material such as a strip of filter paper or polymeric film) as a substantially water-free coating, for example. In certain embodiments, the carrier 16 is made from a water-impermeable material; Thus, in those embodiments, the test microorganisms 17 are enveloped by a wax composition (not shown in FIG. 1) as described herein and, optionally, a water-impermeable substrate, as shown in FIGS. 4A-B and described below.

The self-contained biological indicator 100 includes a liquid medium 20 (e.g., an aqueous liquid medium) disposed in an openable container 18. The contents (e.g., liquid medium 20) of the openable container 18 are in selective communication with the compartment 11 of the housing 10. In the illustrated embodiment of FIGS. 1-3, the openable container 18 is a normally sealed, pressure-openable container, such as a frangible glass ampoule. A person having ordinary skill in the art will recognize other suitable openable containers 18 (including some embodiments wherein the container is disposed outside the housing 10) and means (e.g., valves, burstable seals) of selective fluid communication between the openable container and the compartment.

The self-contained biological indicator 100 includes a nutrient composition that facilitates germination and/or outgrowth of the test microorganisms. The nutrient composition (not shown in FIGS. 1-3) is disposed in the container or the housing. In the illustrated embodiment of FIGS. 1-3, the nutrient composition is dissolved and/or suspended in the liquid medium 20 that is disposed in the openable container 18 disposed in the housing 10. In certain alternative embodiments, the nutrient composition can be disposed in the housing in a dry form (e.g., a dry coating, a powder, a tablet) that can be dissolved and/or suspended in the liquid medium upon actuation of the openable container. Additionally, upon actuation of the openable container, the liquid medium and the nutrient composition can come into contact with the test microorganisms, thereby facilitating the growth (and detection) of any viable test microorganisms, if present, in the biological indicator.

In the illustrated embodiment of FIGS. 1-3, the container 18 is snugly retained within the compartment, so that very little of the volume of the compartment 10 remains unoccupied. The container 18 is separated from the wall 12 of the compartment 10 by the carrier 16, thereby providing cavities 24 and 26 between the wall 12 and the container 18. The open end 14 of the compartment 10 is provided with a gas-transmissive, bacteria-impermeable closure member which is shown as a sheet 22. The sheet 22 may be sealed to the open end 14 of the compartment 10 by e.g., heat or adhesive sealing, or by means of cap 28 (shown removed in FIG. 2) which has an aperture 29 therethrough adjacent the sheet 22. The aperture 29 permits passage of a sterilant, from outside the housing into the housing. The cap 28 can be formed from a variety of materials (e.g., metal, plastic) using processes that are well known in the art. During steam sterilization, the steam permeates the sheet 22 and passes through cavities 24 and 26 to contact the test microorganisms 17 disposed on the carrier 16.

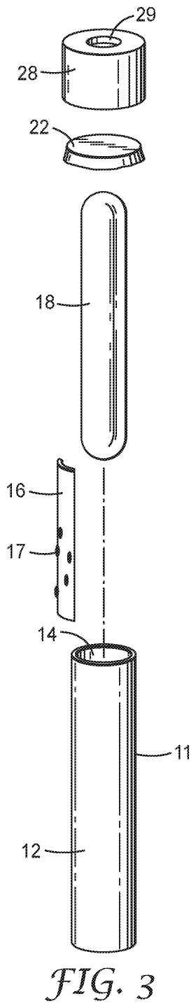

As shown in FIG. 3, the self-contained biological indicator 100 may be easily assembled by sequentially inserting into the open end 14 of the compartment 10 the carrier 16 (on which the test microorganisms 17 are disposed) and the container 18 and sealing the open end 14 of the tube with the sheet 22. Prior to the assembly, the test microorganisms 17 may be deposited onto the carrier 16 as a liquid suspension and subsequently dried before placing the carrier 16 into the container 10.

Optionally, the biological indicator 100 may further include a detector material (e.g., a chromogenic or fluorogenic enzyme substrate and/or a pH indicator; not shown) which is capable of undergoing a visible color change in response to growth of the spores. The detector material may be present in the liquid medium and/or may be present in a dry form (e.g., a powder or a tablet) disposed in the housing 10.

FIG. 4A-B show various views of the carrier 16 of the self-contained biological indicator 100 of FIGS. 1 and 3. Disposed on the carrier 16 is a plurality of test microorganisms 17 and a wax composition 30, both as described herein. When producing a biological indicator of the present disclosure, the test microorganisms 17 can be applied to the carrier 16 suspended in a liquid solvent (e.g., sterile water, not shown). Subsequently, the solvent is removed (e.g., by evaporation) leaving a dry coating of test microorganisms on the carrier. The wax composition 30 can then be applied to the carrier 16, either enveloping the individual test microorganisms in the wax composition or enveloping the test microorganisms between the wax composition and the carrier material.

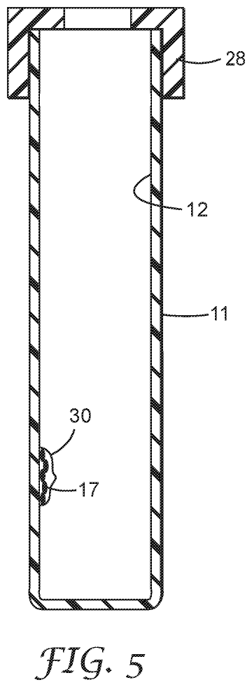

FIG. 5 shows an alternative way to provide the test microorganisms in the housing (comprising compartment 11 and cap 28) of a self-contained biological indicator according to the present disclosure. In this embodiment, rather than providing the test microorganisms in the housing on a carrier (not shown in FIG. 5), the test microorganisms 17 are placed inside the compartment on the inner surface of the at least one wall 12. The test microorganisms 17 and the wax composition 30 can be delivered onto the wall 12 as discussed above for the placement of the test microorganisms and the wax composition onto the carrier. After depositing the test microorganisms 17 and the wax composition 30 in the housing, a container (e.g., frangible glass ampoule, not shown) containing a liquid medium a nutrient composition can be positioned in the housing to produce a self-contained biological indicator.

Sterilization Processes

Biological indicators of the present disclosure may be used to monitor the effectiveness of one or more types of sterilization procedures, including sterilization procedures that use steam (e.g., pressurized steam) as the sterilant.

In at least some of the sterilization processes, an elevated temperature, for example, 121° C., 132° C., 134° C., 135° C. or the like, is included or may be encountered in the process. In addition, elevated pressures and/or a vacuum may be encountered, for example, 15 psi (1×10⁵ Pa) at different stages within a single given sterilization cycle, or in different sterilization cycles.

In the case of steam being the sterilant, the sterilization temperatures can include 121° C., 132° C., 134° C., 135° C. The rapid readout biological indicators are suitable for steam sterilization cycles at each of the temperatures above and for each temperature the cycle can have a different air removal process chosen from gravity, prevacuum (“pre-vac”), and steam flush pressure pulse (SFPP). Each of these cycles may have different exposure times depending on the type of instruments/devices being sterilized. In this disclosure, prevacuum and SFPP are also labeled as Dynamic Air Removal (DAR) cycles.

A tabular representation of exemplary steam sterilization cycles in which the present biological indicators can be used is shown below:

| 121° C. | | | 132° C. | | | 134° C. | | | 135° C. | | |
|---|---|---|---|---|---|---|---|---|---|---|---|
| Gravity | Pre-Vac | SFPP | Gravity | Pre-Vac | SFPP | Gravity | Pre-Vac | SFPP | Gravity | Pre-Vac | SFPP |

In general, a sterilization process includes placing the biological indicator of the present disclosure in a sterilizer. In some embodiments, the sterilizer includes a sterilization chamber that can be sized to accommodate a plurality of articles to be sterilized and can be equipped with a means of evacuating air and/or other gases from the chamber and a means for adding steam to the chamber. The self-contained biological indicator can be positioned in areas of the sterilizer that are most difficult to sterilize. Alternately, the biological indicator can be positioned in process challenge devices to simulate sterilization conditions where steam may not be delivered as directly as would be the case in more favorable sterilization circumstances.

The steam sterilant can be added to the sterilization chamber after evacuating the chamber of at least a portion of any air or other gas present in the chamber. Alternatively, steam can be added to the chamber without evacuating the chamber. A series of evacuation steps can be used to assure that the steam sterilant reaches all desired areas within the chamber and contacts all desired article(s) to be sterilized, including the biological indicator.

The self-contained biological indicators are capable of determining the efficacy of one or more steam sterilization cycles chosen from the powerset of the following eleven cycles: 121 C gravity, 121 C pre-vac, 121 C SFPP, 132 C gravity, 132 C pre-vac, 132 C SFPP, 134 C pre-vac, 134 C SFPP, 135 C gravity, 135 C pre-vac, and 135 C SFPP, preferably within less than 1 hr.

Liquid Medium

A self-contained biological indicator of the present disclosure comprises a liquid medium disposed in an openable container, the contents of the container being in selective fluid communication with the compartment. Suitable containers include, for example, the glass ampoule 18 described in U.S. Pat. No. 3,661,717; the inner container 48 described in U.S. Pat. No. 5,223,401; the inner compartment 18 and inner containers 48 and 78 described in U.S. Pat. No. 6,623,955; and frangible container 120 described in U.S. Patent Application Publication Nos. 2013/0302849.

The liquid medium can contain one or more of the enzyme substrates mentioned herein. In certain embodiments, the enzyme substrate is 4-methylumbelliferyl-alpha-D-glucoside (MUG). In some embodiments, the liquid medium optionally may also include a nutrient composition that facilitates germination and/or outgrowth of the test microorganisms. In some preferred embodiments, the solvent of the liquid medium is water.

Suitable nutrients may be provided in the housing initially in a dry form (e.g., powdered form, tablet form, caplet form, capsule form, a film or coating, entrapped in a bead or other carrier, another suitable shape or configuration, or a combination thereof). When combined with the liquid medium (e.g., when the biological indicator is actuated), the nutrients can contact the test microorganisms and facilitate growth of any viable test microorganisms that remain in the housing after the biological indicator has been exposed to a sterilization process.

The nutrient can include one or more sugars, including, but not limited to, glucose, fructose, dextrose, maltose, trehalose, cellobiose, or the like, or a combination thereof. Alternatively, the nutrients may include complex media, such as peptone, tryptone, phytone peptone, yeast extract, soybean casein digest, other extracts, hydrolysates, etc., or a combination thereof. In other embodiments, the nutrient comprises a combination of one or more complex media components and other specific nutrients. The nutrient can also include a salt, including, but not limited to, sodium chloride, potassium chloride, calcium chloride, or the like, or a combination thereof. In some embodiments, the nutrient can further include at least one amino acid, including, but not limited to, at least one of methionine, phenylalanine, alanine, tyrosine, and tryptophan.

As part of a self-contained biological indicator, the liquid medium; optionally comprising nutrients, an enzyme substrate, and other components; is typically present throughout the sterilization procedure but is kept separate in the actuatable container and not accessible to the test microorganisms until the biological indicator is actuated. After the sterilization process is completed and the biological indicator is used to determine the efficacy of the sterilization, the liquid medium is placed in contact with the test microorganisms and the nutrient resulting in a mixture. In this disclosure, placing the liquid medium in contact with the test microorganisms includes activating the actuatable container so that the liquid medium is released and contacts the test microorganisms. This process may include mixing of the liquid medium with the test microorganisms, such as manual or mechanical shaking of the housing of the biological indicator so that the liquid medium adequately mixes with the test microorganisms.

In this disclosure, the process of bringing the test microorganisms and liquid medium together is referred to as “activation” of the biological indicator. That is, the term “activation” and variations thereof, when used with respect to a biological indicator refer generally to bringing one or more test microorganisms (e.g., spores) into contact with the liquid medium (comprising, e.g., a nutrient medium for the test microorganisms of interest and an enzyme substrate). For example, when an actuatable container within the biological indicator that contains the liquid medium is at least partially fractured, punctured, pierced, crushed, cracked, breaking, or the like, such that the medium has been put in fluid communication with the test microorganisms, the biological indicator can be described as having been “activated.” Said another way, a biological indicator has been activated when the test microorganisms have been contacted with the liquid medium that was previously housed separately from the test microorganisms.

In some preferred embodiments, the mixture resulting from mixing the liquid medium with the test microorganisms after activation remains isolated within the housing of the biological indicator after the sterilization cycle has been completed and no additional reagents or components are added to it during or after activation. If at least one of the test microorganisms is viable and grows, then an enzyme produced by the microorganism can catalyze the cleavage of the enzyme substrate, which can produce the fluorescently detectable compound. This means that the same solution in the same container (housing) is used for three separate events: (a) test microorganism germination/growth, if the test microorganism is viable, (b) the enzymatic cleavage of the enzyme substrate, resulting in the production of the fluorescently-detectable compound, and (c) the fluorescence detection of the fluorescently-detectable compound.

In some embodiments, the liquid medium may comprise a buffered solution such as, for example, the buffered solution described in U.S. Patent Application No. 62/964,369 entitled “SELF-CONTAINED BIOLOGICAL INDICATOR WITH SALT COMPOUND” filed on Jan. 22, 2020;

which is incorporated herein by reference in its entirety. The ionic conditions of the buffered solution should be such that the enzyme and enzyme substrate, if present in the biological indicator, are not affected. In some embodiments, a buffer solution is used as part of the liquid medium, such as phosphate buffers, (e.g., phosphate buffered saline solution, potassium phosphate or potassium phosphate dibasic), tris(hydroxymethyl) aminomethane-HCl solution, or acetate buffer, or any other buffer suitable for sterilization known in the art. Buffers suitable for the present biological indicators should be compatible with fluorogenic and chromogenic enzyme substrates, if such enzyme substrates are used as part of the biological indicator.

The concentration of enzyme substrate, if present in the liquid medium before or after activation of the biological indicator, depends upon the identity of the particular substrate and enzyme, the amount of enzyme-product that must be generated to be detectable, either visually or by instrument, and the amount of time that one is willing to wait in order to determine whether active enzyme is present in the reaction mixture. Preferably, the amount of enzyme substrate is sufficient to react with any residual active enzyme present, after the sterilization cycle, within about an eight-hour period of time, such that at least 10⁻⁸ molar enzyme-modified product is produced. In cases where the enzyme substrate is a 4-methylumbelliferyl derivative, the inventors have been found that its concentration in the aqueous buffered solution is preferably between about 10⁻⁵ and 10⁻³ molar.

In some embodiments, the biological indicator may comprise an additional indicator compound that can facilitate the detection of another metabolic activity of the test microorganisms (e.g., spore) (aside from an enzyme substrate that can produce a fluorescently detectable compound). This additional metabolic activity can also be an enzymatic activity. Non-limiting examples of indicator compounds include a chromogenic enzyme substrate (e.g., observable in the visible spectrum), a pH indicator, a redox indicator, a chemiluminescent enzyme substrate, a dye, and a combination of any two or more of the foregoing indicator compounds.

In some embodiments, the additional indicator is a pH indicator that produces a change in color when the pH decreases, indicating growth of the test microorganisms. In some embodiments, the pH indicator is bromocresol purple. The pH indicator can be used to detect a second biological activity, such as the fermentation of a carbohydrate to acid end products (suggesting survival of the test microorganisms) and an enzymatic biological activity such as α-D-glucosidase enzyme activity, for example. These activities can indicate the presence or absence of a viable test microorganism following the exposure of a biological indicator to a sterilization process, for example. The bromocresol purple can be used at a concentration of about 0.03 g/L in the aqueous mixture, for example. The 4-methylumbelliferyl-α-D-glucoside can be used, for example, at a concentration of about 0.05 to about 0.5 g/L (e.g., about 0.05 g/L, about 0.06 g/L, about 0.07 g/L, about 0.08 g/L, about 0.09 g/L, about 0.1 g/L, about 0.15 g/L, about 0.2 g/L, about 0.25 g/L, about 0.3 g/L, about 0.35 g/L, about 0.4 g/L, about 0.45 g/L, about 0.5 g/L) in the aqueous mixture.

Wax Composition-Wicking Member

A self-contained biological indicator of the present disclosure optionally comprises a wax composition-wicking member. The function of the wax composition-wicking member is to absorb the molten wax composition and/or wick the wax composition away from the test microorganisms while the biological indicator is exposed to a steam sterilization process and to sequester the wax from the test microorganisms when the test microorganisms are contacted with the liquid medium and the nutrient composition to detect viable test microorganisms, if present, in the biological indicator after it has been exposed to the sterilization process. In certain embodiments, wherein the wax composition-enveloped test microorganisms are affixed to a substrate (e.g., a carrier or at least one wall of the housing, as described herein), the wax composition-wicking member can be positioned in contact with the carrier or wall, respectively, in order to draw the wax away as it melts. Additionally, or alternatively, the wax composition-wicking member can be positioned in contact with the wax, so that the wax can flow into or onto the wicking member as it melts.

Materials (e.g., fibrous materials such as filter paper) that serve to wick (e.g., by capillary action) a molten wax composition away from a source are known in the art of time-temperature dosimeters (see, for example, U.S. Pat. No. 9,301,258; which is incorporated herein by reference in its entirety). Suitable materials for the wax composition-wicking member of the present disclosure include, for example, paper (e.g., a strip of filter paper).

Method of Assessing the Efficacy of a Sterilization Process

In another aspect, the present disclosure provides a method for determining the efficacy of a sterilization process. The method comprises positioning a sterilization process biological indicator in a sterilization chamber. The biological indicator comprises a plurality of test microorganisms that are at least partially enveloped by a wax composition. “Sterilization process biological indicator”, as used in reference to a method of the present disclosure, is used broadly to include an article that contains or comprises a plurality of test microorganisms intended to verify the efficacy of a moist-heat sterilization process. Sterilization process biological indicators include, for example, a container holding a substrate (e.g., a strip, a coupon, a bead, or a yarn made of a material known in the art of biological indicators for carrying test microorganisms during a sterilization process) having sterilization process test microorganisms affixed thereto, or an article (e.g., a bead) comprising the aforementioned wax composition and the test microorganisms enveloped thereby.

The wax composition in the sterilization process biological indicators used in the method of the present disclosure comprises a long-chain (greater than C22) linear or branched alkyl or alkenyl alcohol, wherein the alkyl or alkenyl alcohol is not linked through an ester bond or an ether bond to a short chain alkyl or aryl (C1-C6) group, wherein the alkyl or alkenyl alcohol is not linked through an ester bond or an ether bond to a long chain (C8-C36) alkyl or alkenyl group, wherein the alkyl or alkenyl alcohol is not linked through an amide bond to a linear or branched long chain (C8-C36) alkyl or alkenyl amine or acid. The wax composition has a melting point from 78° C. to 120° C.

In certain embodiments, positioning the sterilization process biological indicator in a sterilization chamber comprises positioning the biological indicator so that the wax composition, when it reaches its melting point, will flow away from the test microorganisms. For example, if the test microorganisms are disposed on a carrier (as shown in FIG. 4A) and a first edge of the carrier is positioned higher in the biological indicator than a second edge then, as the wax composition melts, gravity will tend to draw the wax composition toward the second edge (and away from at least some of the test microorganisms).

The method further comprises, while the indicator is positioned in the sterilization chamber, exposing the biological indicator to moist heat at a temperature of at least 121° C. In certain embodiments, exposing the biological indicator to moist heat at a temperature of at least 121° C. can comprise processing the biological indicator in an automated steam sterilizer using a preprogrammed sterilization cycle selected from the powerset of the following eleven cycles: 121 C gravity, 121 C pre-vac, 121 C SFPP, 132 C gravity, 132 C pre-vac, 132 C SFPP, 134 C pre-vac, 134 C SFPP, 135 C gravity, 135 C pre-vac, and 135 C SFPP.

After exposing the biological indicator to the moist heat, the method comprises contacting the test microorganisms with a detection medium. The detection medium typically comprises an aqueous liquid that includes a reagent that facilitates detection of a viable test microorganism. The reagent can be an effective quantity of any reagent known in the art to cause the test microorganism to reproduce to a detectable extent (e.g., by turbidity) or to carry out one or more reactions (e.g., an enzyme-catalyzed reaction) to a detectable extent (e.g., by chromogenic, fluorogenic, or chemiluminescent detection methods).

In certain embodiments, contacting the test microorganisms with a detection medium comprises adding the detection medium (e.g., by pipette) to a housing or other vessel in which the test microorganisms are disposed. In certain embodiments, contacting the test microorganisms with a detection medium comprises activating a biological indicator (e.g., by opening a frangible container containing the medium in the biological indicator) to cause contact between the medium, the detection reagent, and the test microorganisms. Optionally, after placing the test microorganisms in liquid contact with the detection medium, the components can be mixed (e.g., by manual or mechanical agitation or vortex action).

After contacting the test microorganisms with the detection medium, the method comprises incubating the indicator at a predetermined temperature for a period of time sufficient to detect a presence of one of the test microorganisms, if viable. The predetermined temperature can be any suitable incubation temperature described herein for the test microorganism and/or the enzyme activity thereof. The period of time can be any suitable period of time of incubation known for detecting microorganisms or enzyme activities thereof. In certain embodiments, the specified period of time is less than 8 hours, in some embodiments, less than 1 hour, in some embodiments, less than 30 minutes, in some embodiments, less than 15 minutes, in some embodiments, less than 5 minutes, and in some embodiments, less than 1 minute. In other embodiments, the suitable incubation time for the biological indicator of this disclosure is from 10 minutes to 4 hours, or from 10 min to 1 hr, or from 10 min to 50 min, or from 10-30 min, or from 10-20 min, or from 10-25 min, or from 15 to 30 min, or from 15-25 min, or from 15-20 min.

During and/or after incubating the mixture for the period of time, the viability of at least one test microorganism in the biological indicator can be detected using procedures known in the art including visual detection and/or automated detection. Detecting a fluorescent product of an enzyme reaction, for example, comprises directing electromagnetic radiation (e.g., radiation within the ultraviolet spectrum of electromagnetic energy) into the mixture and detecting electromagnetic radiation (e.g., radiation within the ultraviolet spectrum or visible spectrum of electromagnetic energy) emitted by the fluorescent product in the mixture, as described herein. In certain embodiments, detecting electromagnetic radiation emitted by the fluorescent product comprises detecting electromagnetic radiation using an automated detector (e.g., an auto-reader as described herein).

In any of the above embodiments, the method further can comprise prior to the exposing the indicator to the steam sterilization process, positioning an article to be sterilized in the sterilization chamber.

In any embodiment of the method, detecting a fluorescent product comprises detecting a quantity of fluorescence emitted by the fluorescent product. In any embodiment, the quantity of fluorescence detected can be compared to a threshold quantity. In any embodiment, a first quantity of fluorescence detected after a first specified time period can be compared to a second quantity of fluorescence detected after a second specified time period. In certain embodiments, detecting at least a threshold quantity of the fluorescent product or detecting a threshold quantity of test microorganisms (e.g., by turbidity) indicates a lack of efficacy of the sterilization process.

In any embodiment of the method, positioning a sterilization process biological indicator in a sterilization chamber can comprise positioning a sterilization process biological indicator comprising a housing with the test microorganisms therein. In these embodiments, contacting the test microorganisms with a detection medium comprises a contacting the test microorganisms with the detection medium inside the housing. In these embodiments, wherein contacting the test microorganisms with a detection medium can comprise contacting the test microorganisms with an aqueous medium comprising a detection reagent. The detection reagent can be selected from the group consisting of a nutrient, a pH indicator, a redox indicator, a fluorogenic enzyme substrate, a chromogenic enzyme substrate, and a combination of any two or more of the foregoing detection reagents.

To detect a detectable change caused by a viable test microorganism, the biological indicator can be assayed immediately after the liquid medium and the test microorganisms have been combined to achieve a baseline reading. After that, any detectable change from the baseline reading can be detected. The biological indicator can be monitored and measured continuously or intermittently. In some embodiments, a portion of, or the entire, incubating step may be carried out prior to measuring the detectable change. In some embodiments, incubation can be carried out at one temperature (e.g., at 37° C., at 50-60° C., etc.), and measuring of the detectable change can be carried out at a different temperature (e.g., at room temperature, 25° C., or at 37° C.). In other embodiments, the incubation and measurement of fluorescence occurs at the same temperature.

The readout time of the biological indicator (i.e., the time to determine the effectiveness of the sterilization process) can be, in some embodiments, less than 8 hours, in some embodiments, less than 1 hour, in some embodiments, less than 30 minutes, in some embodiments, less than 15 minutes, in some embodiments, less than 5 minutes, and in some embodiments, less than 1 minute. In other embodiments, the readout time for the biological indicator of this disclosure is from 10 min to 1 hr, or from 10 min to 50 min, or from 10-30 min, or from 10-20 min, or from 10-25 min, or from 15 to 30 min, or from 15-25 min, or from 15-20 min. The detection of fluorescence above the baseline reading that would indicate presence of viable spores (i.e., a failed sterilization process) can be performed according to any method know in the art, including area under curve (in a plot of time vs fluorescence intensity), monitoring a change in slope of the curve, using a threshold value for the fluorescence, etc., or a combination thereof of two or more techniques.

One of the advantages of the biological indicators of this disclosure is that a single type can be used for various steam sterilization conditions. The working examples below show a single type of biological indicator can be used for all of the following steam sterilization cycles: 121° C. gravity, 121° C. pre-vac, 121° C. SFPP, 132° C. gravity, 132° C. pre-vac, 132° C. SFPP, 134° C. pre-vac, 134° C. SFPP, 135° C. gravity, 135° C. pre-vac, and 135° C. SFPP. For that reason, the biological indicator can be used for any subset of cycles chosen from the set above. That is, a single biological indicator is capable of determining the efficacy of one or more sterilization cycles chosen from the powerset of 121° C. gravity, 121° C. pre-vac, 121° C. SFPP, 132° C. gravity, 132° C. pre-vac, 132° C. SFPP, 134° C. pre-vac, 134° C. SFPP, 135° C. gravity, 135° C. pre-vac, and 135° C. SFPP.

Kits

In another aspect, the present disclosure provides a kit that can be used for determining the efficacy of a sterilization process. The kit can comprise a plurality of test microorganisms, wherein the test microorganisms are at least partially enveloped by a wax composition, and instructions for using the test microorganisms to assess the efficacy of a steam sterilization process. The wax composition comprises a long-chain (greater than C22) linear or branched alkyl or alkenyl alcohol, wherein the alkyl or alkenyl alcohol is not linked through an ester bond or an ether bond to a short chain alkyl or aryl (C1-C6) group, wherein the alkyl or alkenyl alcohol is not linked through an ester bond or an ether bond to a long chain (C8-C36) alkyl or alkenyl group, wherein the alkyl or alkenyl alcohol is not linked through an amide bond to a linear or branched long chain (C8-C36) alkyl or alkenyl amine or acid. The wax composition has a melting point between 78° C. and 120° C.

In any embodiment, the kit further can comprise a detection reagent selected from the group consisting of a nutrient, a pH indicator, a redox indicator, a fluorogenic enzyme substrate, a chromogenic enzyme substrate, and a combination of any two or more of the foregoing detection reagents. In any of the above embodiments, the kit further can comprise a housing dimensioned to contain the test microorganisms or the detection reagent. In certain embodiments of the kit, the test microorganisms and the detection reagent can be disposed in the housing. In certain embodiments, the wax composition has a melting point of at least about 78 degrees C., at least about 91 degrees C., at least about 99 degrees C., or at least about 105 degrees C.

In any embodiment of the kit, the test microorganisms are completely enveloped by the wax composition. In any embodiment, the kit can comprise any embodiment of the self-contained biological indicator of the present disclosure.

### EXAMPLES

### Examples 1-4. Assembly of a Self-Contained Sterilization Process Biological Indicator

The wax compositions used in this Example are listed in Table 1. Wax compositions were obtained from Baker Hughes (Houston, Tex.). The wax compositions were heated to just above the melting point before depositing them on the spore strips as described below.

TABLE 1

| Example No. | Wax Name | Melting Point (° C.) |
|---|---|---|
| 1 | UNILIN ™ 350 Alcohol | 78 |
| 2 | UNILIN ™ 425 Alcohol | 91 |
| 3 | UNILIN ™ 550 Alcohol | 99 |
| 4 | UNILIN ™ 700 Alcohol | 105 |
| Control | None | N/A |

3M™ ATTEST™ Super Rapid Readout Biological Indicators (part number 1492V) were obtained from 3M Company (St. Paul, Minn.). Those self-contained biological indicators are assembled as shown in FIGS. 4-6 of U.S. Patent Application Publication No. US 2014/0349335, which is incorporated herein by reference in its entirety. The caps of the biological indicators were removed and the contents (glass ampule, insert, and the spore reservoir) were removed. The inside of the tubes and the outside of the glass ampules and the inserts were rinsed with sterile deionized water. 100 microliters of molten wax composition was added by micropipette to the spore reservoir, completely overlying the spores dried on the concave surface of the spore reservoir. The biological indicators were reassembled. Commercially-available ATTEST 1492V biological indicators were used as controls.

### Example 5. Detection of Viable Spores after Exposure to a 135° C. Steam Sterilization Process

Five of each biological indicator of Examples 1-4 and five control biological indicators were placed into the sterilization chamber of a steam resistometer (H&W 101 steam resistometer, available from H&W Technology LLC, Rochester, N.Y.) and were exposed to a Dynamic Air Removal steam sterilization process (135° C. for 3.5 minutes). After exposure to the process, all of the biological indicators were actuated by fracturing the glass ampoule containing the detection medium in the biological indicator. After they were actuated, the biological indicators were incubated in a biological indicator autoreader available from 3M Company (St, Paul, Minn.). The autoreader indicated positive fluorescence was detected within 24 minutes of incubation in every biological indicator. After 7 days of incubation at 60° C., the detection medium was visually observed for a color change due to acid production in the biological indicators. A pH change (as observed by a change in the pH indicator from purple to yellow) was observed in every one of the biological indicators. The data show that the biological indicators with the wax compositions overlaying the spores had the same responses (fluorescent-positive, growth-positive) to the 135° C. steam sterilization process as the control biological indicators.

### Example 6. Detection of Viable Spores after Exposure to a 121° C. Steam Sterilization Process

Five of each biological indicator of Examples 1-4 and five control biological indicators were placed into the sterilization chamber of a steam resistometer (H&W 101 steam resistometer, available from H&W Technology LLC, Rochester, N.Y.) and were exposed to a Dynamic Air Removal steam sterilization process (121° C. for 10 minutes). The biological indicators were held in a rack with the caps uppermost. In this position, the spore reservoirs were canted, relative to the vertical axis of the biological indicators. Accordingly, it was noticed after the biological indicators were processed in the steam resistometer, at least some of the wax composition had dripped out of the spore reservoir and into the bottom of the housing.

After exposure to the process, all of the biological indicators were actuated by fracturing the glass ampoule containing the detection medium in the biological indicator. After they were actuated, the biological indicators were incubated in a biological indicator autoreader available from 3M Company (St, Paul, Minn.) and both fluorescence (enzyme-based early readout) and growth (spore viability) parameters were tested as described in Example 5. The results are shown in Table 2. The data indicate that the resistance of the spores (as indicated by both the fluorescence and growth measurements) generally increased in the biological indicators that included wax compositions with melting points greater than or equal to 91° C. The biological indicators with a wax composition having a melting point of 78° C. showed higher resistance of spore viability to the 121° C. process, but not higher resistance of the spore-associated enzyme activity.

TABLE 2
Effect of 121° C. steam sterilization process on the
rapid (fluorescent) readout and 7 days growth readout in
biological indicators comprising spores enveloped by a wax
composition. Five biological indicators were tested per condition.

| Biological | Number of Fluorescent- | Number Growth- |
|---|---|---|
| Indicator | Positive | Positive |
| Control | 0/5 | 0/5 |
| Example 1 | 0/5 | 4/5 |
| Example 2 | 3/5 | 3/5 |
| Example 3 | 4/5 | 5/5 |
| Example 4 | 5/5 | 5/5 |

Various modifications and alterations to this invention will become apparent to those skilled in the art without departing from the scope and spirit of this invention. It should be understood that this invention is not intended to be unduly limited by the illustrative embodiments and examples set forth herein and that such examples and embodiments are presented by way of example only with the scope of the invention intended to be limited only by the claims set forth herein

1. A self-contained sterilization process biological indicator, comprising:
a housing having at least one liquid-impermeable wall that forms an opening into a compartment;
a plurality of test microorganisms disposed in the housing, wherein the test microorganisms are at least partially enveloped by a wax composition;
wherein the wax composition comprises a long-chain (greater than C22) linear or branched alkyl or alkenyl alcohol;
wherein the alkyl or alkenyl alcohol is not linked through an ester bond or an ether bond to a short chain alkyl or aryl (C1-C6) group;
wherein the alkyl or alkenyl alcohol is not linked through an ester bond or an ether bond to a long chain (C8-C36) alkyl or alkenyl group;
wherein the alkyl or alkenyl alcohol is not linked through an amide bond to a linear or branched long chain (C8-C36) alkyl or alkenyl amine or acid;
wherein the wax composition has a melting point between 78° C. and 120° C.;
a liquid medium disposed in an openable container, the contents of the container being in selective fluid communication with the compartment; and
a nutrient composition that facilitates germination and/or outgrowth of the test microorganisms, wherein the nutrient composition is disposed in the container or the housing;
wherein the housing comprises an opening that permits passage of a sterilant, from outside the housing into the housing.

2. The self-contained sterilization process biological indicator of claim 1, wherein the container is disposed in the housing.

3. The self-contained sterilization process biological indicator of claim 1, wherein the nutrient composition is disposed in the container.

4. The self-contained sterilization process biological indicator of claim 1, further comprising a detection reagent disposed in the housing.

5. The self-contained sterilization process biological indicator of claim 4, wherein the detection reagent is disposed in the container.

6. The self-contained sterilization process biological indicator of claim 1, wherein the test microorganisms are affixed to a carrier, wherein the test microorganisms are enveloped by the wax composition and/or the carrier.

7. The self-contained sterilization process biological indicator of claim 6, wherein the carrier is moisture-impermeable.

8. The self-contained sterilization process biological indicator of claim 1,
wherein the test microorganisms are affixed to a portion of the wall of the housing, wherein the test microorganisms are enveloped by the wax composition and/or the portion of the wall.

9. The self-contained sterilization process biological indicator of claim 1, wherein the wax composition has a melting point of at least about 78 degrees C.

10. The self-contained sterilization process biological indicator of claim 9, wherein the wax composition has a melting point of at least about 91 degrees C.

11. The self-contained sterilization process biological indicator of claim 10, wherein the wax composition has a melting point of at least about 99 degrees C.

12. The self-contained sterilization process biological indicator of claim 11, wherein the wax composition has a melting point of at least about 105 degrees C.

13. The self-contained sterilization process biological indicator of claim 1, further comprising a wax composition-wicking member disposed in the housing.

14. The self-contained sterilization process biological indicator of claim 13, wherein the wax composition-wicking member contacts the carrier.

15. The self-contained sterilization process biological indicator of claim 13, wherein the wax composition-wicking member contacts the wall of the housing.

16. The self-contained sterilization process biological indicator of claim 13, wherein the wax composition-wicking member contacts the wax composition.

17. The self-contained sterilization process biological indicator of claim 13, wherein the wax composition-wicking member comprises a fibrous material.

18. A method of determining effectiveness of a sterilization process, the method comprising:
positioning a sterilization process biological indicator in a sterilization chamber;
wherein the biological indicator comprises a plurality of test microorganisms that are at least partially enveloped by a wax composition;
wherein the wax composition comprises a long-chain (greater than C22) linear or branched alkyl or alkenyl alcohol;
wherein the alkyl or alkenyl alcohol is not linked through an ester bond or an ether bond to a short chain alkyl or aryl (C1-C6) group;
wherein the alkyl or alkenyl alcohol is not linked through an ester bond or an ether bond to a long chain (C8-C36) alkyl or alkenyl group;
wherein the alkyl or alkenyl alcohol is not linked through an amide bond to a linear or branched long chain (C8-C36) alkyl or alkenyl amine or acid;
wherein the wax composition has a melting point between 78° C. and 120° C.;
while the indicator is positioned in the sterilization chamber, exposing the biological indicator to a moist heat at a temperature of at least 121 degrees C.;
after exposing the biological indicator to the moist heat, contacting the test microorganisms with a detection medium;
after contacting the test microorganisms with the detection medium, incubating the indicator at a predetermined temperature for a period of time sufficient to detect a presence of one of the test microorganisms, if viable.

19. The method of claim 18, wherein positioning a sterilization process biological indicator in a sterilization chamber comprises positioning a sterilization process biological indicator comprising a housing with the test microorganisms therein, wherein contacting the test microorganisms with a detection medium comprises a contacting the test microorganisms with the detection medium inside the housing.

20. The method of claim 19, wherein contacting the test microorganisms with a detection medium comprises contacting the test microorganisms with an aqueous medium comprising a detection reagent selected from the group consisting of a nutrient, a pH indicator, a redox indicator, a fluorogenic enzyme substrate, a chromogenic enzyme substrate, and a combination of any two or more of the foregoing detection reagents.

21-33. (canceled)
